(19) United States
(12) Patent Application Publication
(10) Pub. No.: US 2009/0248053 A1
(43) Pub. Date: Oct. 1, 2009

(54) MEDICAL INSTRUMENT WITH A FLEXIBLE INSERT

(76) Inventors: Uwe Bacher, Tuttlingen (DE); Sabine Summerer, Tuttlingen (DE)

Correspondence Address: ST. ONGE STEWARD JOHNSTON & REENS, LLC, 986 BEDFORD STREET, STAMFORD, CT, 06905-5619, US

(21) Appl. No.: 12/415,135

(22) Filed: Mar. 31, 2009

(30) Foreign Application Priority Data
Mar. 31, 2008 (DE) 10 2008 017 298.7

(51) Int. Cl. A61B 17/32

(52) U.S. Cl. 606/170

(57) ABSTRACT

A medical instrument comprises a flexible shaft which has a bendable distal end and extending along said shaft, a flexible insert which at a distal end has a tool. The flexible insert has at its proximal end a connection piece connected releasably to a grip part of a handle of the instrument. The flexible insert is secured in an axially immovable manner to a distal end of the flexible shaft and is connected to the grip part at the proximal end in an articulated manner and held in place by a catch.

### BACKGROUND OF THE INVENTION

The invention relates to a medical instrument with a flexible shaft which has a bendable distal end and, extending along the flexible shaft, a flexible insert which at its distal end has a tool and at its proximal end has a connection piece connected to a grip part, which is mounted on a handle, arranged at the proximal end of the shaft, so as to pivot about a pivot axis.

Medical instruments of this kind in the form of dissecting forceps and gripping forceps, and also in the form of scissors, punches and pincers for biopsy, are known from sections 4 and 5 of the catalogue of the company Karl Storz GmbH & Co. KG, Tuttlingen, Germany, Laparoskopie, 5th edition 1/2005.

These medical instruments are composed of three main structural parts, namely the shaft, the handle with a movable grip part, and the insert pushed into the shaft. At the distal end, the insert carries a tool designed for the intended field of application, e.g. as gripper parts in the case of dissecting and gripping forceps, and as cutting blades in the case of scissors, punches and pincers for biopsy. The tool can be designed as an electrode and be used for coagulation. The proximal end of the insert is connected releasably to the movable grip part. A movement of the grip part causes an axial reciprocating movement of an actuating element of the insert and, for example, causes the opening or closing of jaw parts of the tool. The insert is usually fitted by being pushed into the shaft from the distal direction until a limit stop on the tool strikes against the distal end of the shaft. The insert is fastened in place at the proximal end of the shaft in most cases by a screw connection. The axially displaceable part of the insert protruding from the proximal end of the shaft serves as a connection piece for connection to the grip part. In the case of rigid inserts, the click-line connection technique has become established (see DE 197 220 62), in which a spherical end of the connection piece is pushed into a socket in the movable grip part, in an extreme position of pivoting of the grip part, which is then pivoted into the actual working position, in which the spherical end is then locked against escape from the socket.

In medical instruments with a flexible shaft, it has been found that the curvature of a long shaft, and of an optionally bendable end of the shaft, causes axial shifting of the relative position between the shaft and the insert received therein, with the result that the insert is pushed out axially to a slight extent from the distal end. This can be explained by the fact that the insert, despite its flexibility, has to have a certain degree of stiffness in order to be able to transmit the axially directed forces for opening and closing the jaw parts. The fact that the hollow shaft surrounds this insert means that its curvature is necessarily slightly different than the curvature of the insert received in it, resulting in these axial displacements which, however slight they might be, are undesired. In the event of extreme displacements, safe handling of the tool would no longer be guaranteed.

In devices with a flexible shaft and with an additional bending of the shaft, it is necessary to arrange control wires in the shaft in order to ensure the bending of the shaft. These control wires have to be routed out at the proximal end of the shaft and fitted on corresponding control elements. In flexible shafts, therefore, it has become customary practice that the proximal end of the insert, that is to say the connection piece to the movable grip part, is not guided to the connection site directly in the longitudinal axis of the shaft, but instead to a site located to the side. This creates problems in the area of connection of the proximal end of the insert to the grip part since, during movement of the movable grip part, certain excursion movements or pivoting movements of the proximal end area of the insert are needed, since length compensation may be necessary here. This eventually leads to a situation where a pushing of this proximal end section of the insert causes it to be moved distally out of the shaft. This bears the risk that contamination can enter the shaft from the distal end.

It is therefore object of the present invention to ensure operationally reliable fastening of the insert.

### SUMMARY OF THE INVENTION

According to the invention, this object is achieved by a medical instrument comprising a flexible shaft having a bendable distal end, a handle arranged at a proximal end of said flexible shaft, a flexible insert extending along said flexible shaft, said flexible insert having a tool at its distal end and a connection piece at its proximal end, said connection piece being releasably connected to a grip part mounted on said handle, said grip part being mounted pivotally about a pivot axis, wherein said flexible insert is mounted in an axially immovable manner at said bendable distal end of said shaft, and wherein said connection piece of said flexible insert is connected to said grip part in an articulated manner.

The measure whereby the distal end of the insert is fastened in an axially immovable manner on the flexible shaft ensures that the tool, i.e. that part of the insert protruding from the distal end of the shaft, can no longer be moved axially out of the shaft.

The articulated connection of the insert to the grip part at the proximal end permits, during pivoting of the grip part, excursion movements of the portion between the site of attachment to the grip part and the site of entry into the shaft.

Although both measures in combination lead to optimal fixing of the position of the insert, each individual measure taken on its own already makes a substantial contribution to the fixing of the position as such.

In a further embodiment of the invention, the distal end of the insert is fastened on the flexible shaft by a screw fastening.

This measure has the advantage that the position can be safely fixed by means of a screw connection which is of a simple design and is easy to produce. It can also be easily undone, in order to disassemble and clean the medical instrument.

In a further embodiment of the invention, the distal end of the insert is fastened on the flexible shaft by a lock mechanism.

This is very simple from the handling point of view, since the insert simply has to be pushed into the shaft from the distal end until the lock engages. To disassemble the instrument, it is then necessary for the lock connection to be able to be released.

In a further embodiment of the invention, the connection piece at the proximal end has a spherical shape.

This measure is known per se and has the advantage that the articulated connection to the movable grip part can be established easily and safely.

In a further embodiment of the invention, the connection piece is held in the grip part by means of a catch.

This measure has the advantage of the catch ensuring that the connection piece is held in place on the grip part but still permits the necessary articulated movement.

In a further embodiment of the invention, the catch can be moved from a locking engagement with the connection piece to a non-locking position.

This measure has the advantage that the connection between the proximal end of the insert and the grip part can be established or undone by a movement of the catch.

In a further embodiment of the invention, the catch is an axial pin which extends transverse to the longitudinal direction of the medical instrument and which has a recess through which the connection piece can be moved in the non-locking position.

This measure has the advantage that such a pin can be arranged ergonomically, thereby making the process of connection and disconnection easy to carry out, namely by moving the pin. By provision of the recess, the connection piece can be moved past the catch in one position, while this is prevented in the other position, such that the spherical end cannot be moved out past the catch and thus detached from the grip part.

In a further embodiment, the movable grip part has an opening via which the connection piece can be pushed in.

This measure has the advantage that assembly is very easy to perform, namely by pushing the proximal end of the insert, i.e. the connection piece, into the opening in the movable grip part, the catch being located in its non-locking position, after which, by displacement of the catch, the locking connection is established.

In a further embodiment of the invention, the catch is held in the grip part by a retainer and is pretensioned against this retainer by a spring.

This measure has the advantage that the catch is pressed into a well-defined position, expediently into the locking position, and is retained there. The pretensioning force of the spring ensures that the catch is always pressed into this locking position.

In a further embodiment of the invention, the catch has a knob which protrudes from the retainer and via which the catch can be moved from the outside.

This measure has the advantage that the catch can be pressed in from the outside via the knob and can thus be brought into the non-locking position in which the connection piece of the insert can now either be pushed in or drawn out, depending on whether assembly or disassembly is being performed. Thereafter, the knob is released again and the catch is then pressed automatically into the locking position such that, with the connection part then pushed in, the latter is locked in place in the grip part.

In a further embodiment of the invention, a portion of the insert arranged distally in front of the connection piece and having a smaller diameter can be inserted into a groove connected to the opening and can be guided laterally out of the grip part via this groove, the connection piece being larger than the width of the groove.

This measure has the considerable advantage that the portion of smaller diameter can be guided laterally out of the grip part via this lateral groove. The connection piece of greater diameter is prevented from moving out of this lateral groove, so as to ensure that the proximal end of the insert cannot escape via this lateral groove from the grip part. Nonetheless, the groove permits pivoting movements of the insert along the groove, such that the relative movements required between the grip part and the end portion of the insert protruding proximally from the shaft are made possible. This is additionally promoted by the design of the connection piece in the shape of a spherical head, but at the same time the catch ensures a secure hold.

It will be appreciated that the aforementioned features and the features still to be explained below can be used not only in the respectively cited combination but also in other combinations or singly, without departing from the scope of the present invention.

### BRIEF DESCRIPTION OF THE DRAWINGS

The invention is described and explained in more detail below on the basis of a number of selected illustrative embodiments and with reference to the attached drawings, in which:

FIG. 1 shows a side view of a medical instrument with a bendable shaft,

FIG. 1a shows the medical instrument of FIG. 1 from the proximal direction, i.e. as seen by the operating surgeon,

FIG. 1b shows a perspective view of the medical instrument,

FIG. 2 shows a partial side view of the medical instrument of FIG. 1, from the opposite side,

FIG. 3 shows a partial side view of the medical instrument of FIG. 1, with the housing of the handle opened,

FIG. 4 shows a partial detail of the handle to illustrate the locking of a bend control mechanism,

FIG. 4a shows the detail from FIG. 4, with a sectional view through a control element of the bend control mechanism in the locked state,

FIG. 5 shows the detail as in FIG. 4a, in the state when not locked,

FIG. 6 shows the control element of the bend control mechanism in a perspective view on its own,

FIG. 6a shows a view of the control element from FIG. 6 along the arrow 93 in FIG. 6,

FIG. 7 shows an actuating element of the bend control mechanism in a perspective view on its own,

FIG. 8 shows a perspective view of a friction element for locking the bend control mechanism,

FIG. 9 shows a detail view as in FIG. 4, with control wires extending about a drum,

FIG. 10 shows a side view of the drum from FIG. 9,

FIG. 10a shows a cross section along the line Xa-Xa in FIG. 10,

FIG. 11 shows a side view of a flexible insert for the medical instrument in FIG. 1,

FIG. 12 shows an enlarged detail view of the distal end of the tool of the flexible insert,

FIG. 13 shows a side view of a grip part of the instrument shown in FIG. 1,

FIG. 13a shows a view of the grip part seen from the direction of the arrow 134 in FIG. 13,

FIG. 14 shows an enlarged partial cross section along the line XIV-XIV in FIG. 13, with the end of the flexible insert from FIG. 11 in the locked state,

FIG. 14a shows a view corresponding to FIG. 14, with the end of the flexible insert released,

FIG. 15 shows an enlarged partial cross section along the line XV-XV in FIG. 13a, with the end of the flexible insert from FIG. 11 engaged,

FIG. 16 shows a perspective view of a catch on its own,

FIG. 17 shows a detail view of a medical instrument in the area of the grip part in order to illustrate the lock connection,

FIG. 18 shows a view corresponding to FIG. 17, with the lock connection released,

FIG. 19 shows a view corresponding to FIG. 17, as a cross section seen in the viewing plane and with the lock connection deactivated, and

FIG. 20 shows a view corresponding to FIG. 19, with the lock connection activated.

### DETAILED DESCRIPTION OF A PREFERRED EMBODIMENT

A medical instrument shown in the figures is designated in its entirety by reference sign 10.

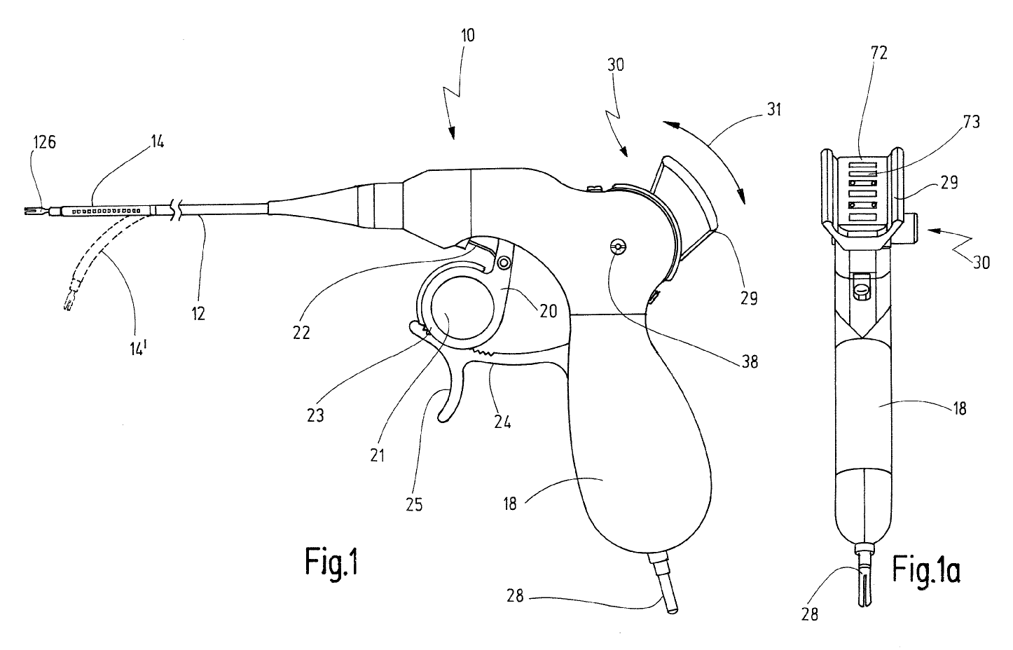

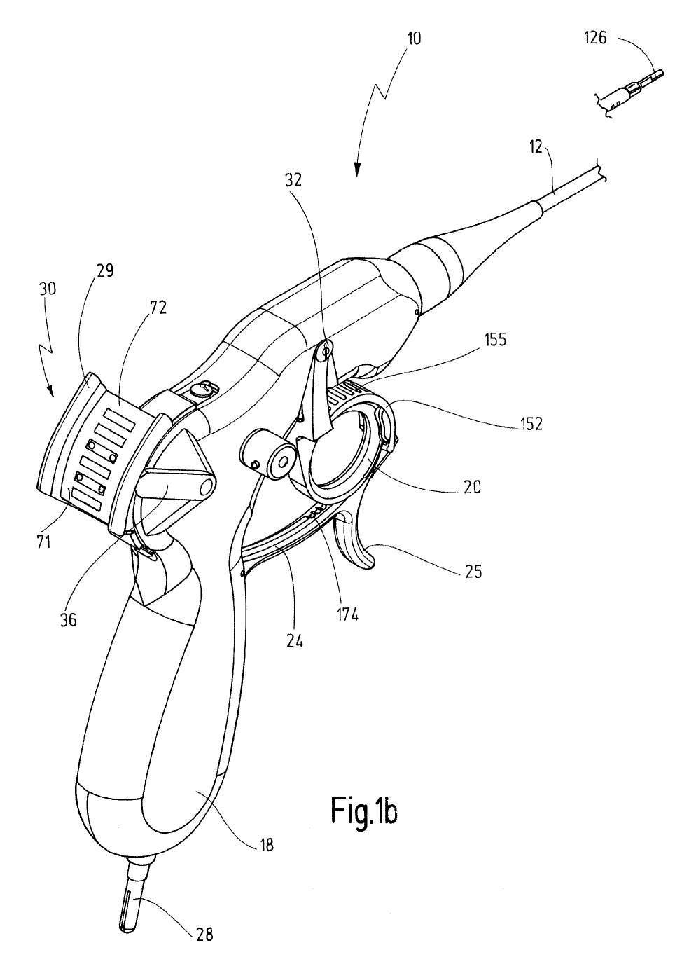

The medical instrument 10 as shown in FIG. 1 comprises a flexible shaft 12 which has a bendable area 14 at its distal end. A tool 126 is arranged distally on the area 14. The tool 126 constitutes a distal end of an insert 22 shown in FIG. 11. The proximal end of the shaft 12 is connected to a handle 18.

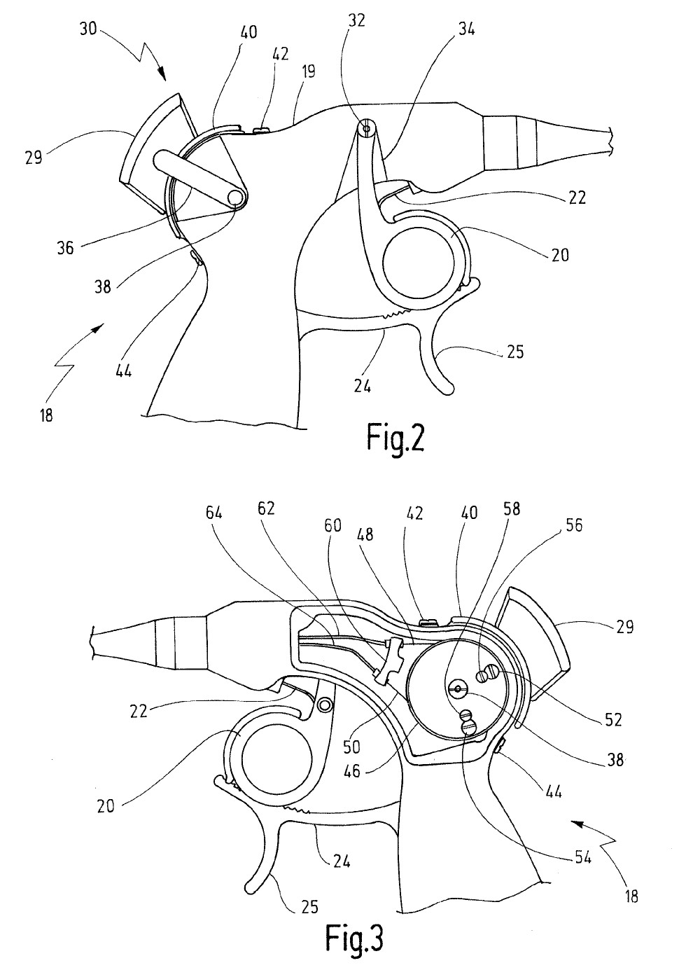

The handle 18 in turn comprises a movable grip part 20. The latter has a round opening 21 which is delimited by a ring portion 23 and through which preferably the index finger of the operating surgeon can be guided in order to execute a movement of the grip part 20, which is pivotable about the pivot axis 32 shown in FIG. 2. The grip part 20 is connected to the proximal end of the insert 22. By virtue of the connection of the grip part 20 to the insert 22, it is operatively connected to the tool 126 and thus serves to actuate the latter, e.g. to open and close a jaw part.

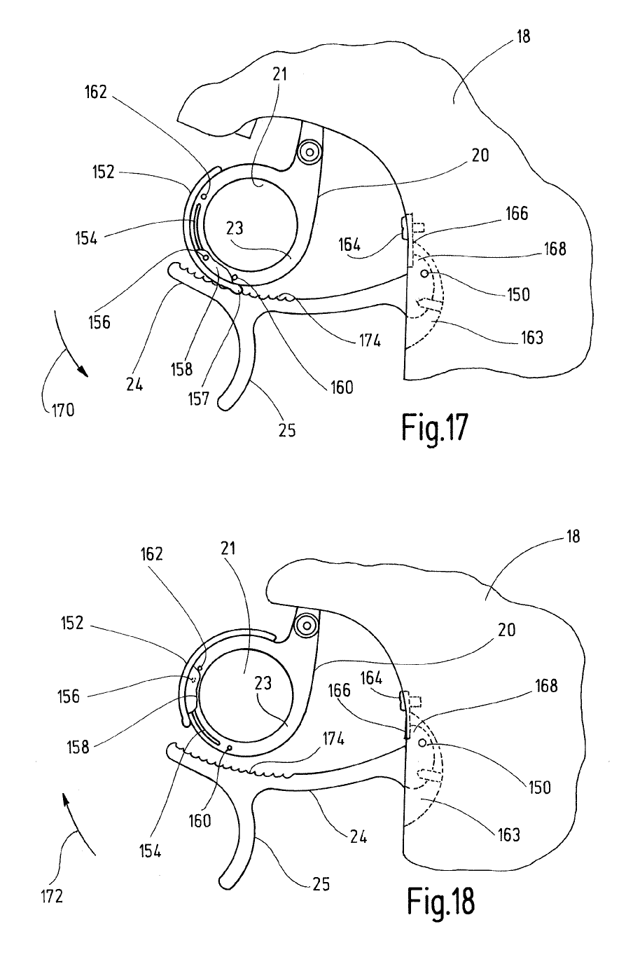

Moreover, the grip part 20 can be brought into contact with a lock 24 that can prevent unwanted movement of the grip part 20 in a distal direction. To permit a release of the lock connection, the lock 24 has, among other things, an arc-shaped attachment 25 which permits a pivoting movement of the lock 24 by the operating surgeon, preferably with the middle finger, as is described in connection with FIG. 17 et seq.

Moreover, the handle 18 is provided with a control element 29 of a bend control mechanism 30, the movement of which in the directions of the double arrow 31 about a pivot axis 38, running perpendicular to the illustrated axis of the shaft 12, permits control of the bending of the bendable end 14 of the shaft 12. An example of the direction of bending is indicated in FIG. 1 by the angled end 14′.

FIG. 1a is a view looking at the control element 29 of the bend control mechanism 30 and at an actuating element 71 in the form of a trigger 72 located thereon. The trigger 72 can be actuated by a thumb of the operating surgeon, as a result of which a movement of the control element 29 is permitted. To provide better grip, grooves 73 are arranged for this purpose on the trigger 72.

The instrument 10 also has a current attachment 28, which can be used, for example, to supply current to optional coagulation inserts.

FIG. 2 indicates the range of pivotability of the grip part 20 about the pivot axis 32 in the area of a recess 34. The control element 29 is connected to the pivot axis 38 via a connecting arm 36.

Between a housing 19 of the handle 18 and the control element 29, there is a friction element in the form of a friction plate 40, which is fastened to the outer face of the handle 18 by screws 42 and 44. As will be described in more detail below, this friction plate 40 is used to stop the bend control mechanism 30 in a defined position.

A drum 46 shown in FIG. 3 is secured on the pivot axis 38 and is thus operatively connected to the control element 29 of the bend control mechanism 30 via the connecting arm 36. A corresponding actuation of the control element 29 thus also results in movement being transferred directly to the drum 46. Two control wires 48 and 50 extending through the shaft 12 from the bendable end 14 of the shaft 12 end on the drum 46, said wires 48 and 50 each extending to the sides of the pivot axis 38 and, in this illustrative embodiment, being fastened on the drum by fastening screws 52 and 54 in combination with securing screws 56 and 58. For this purpose, the control wires 48 and 50, emerging proximally from sleeves 62 and 64, are conveyed through a guide 60 to the drum 46. The control wires 48 and 50 are the actuating elements for the bendable end 14. Together with the drum 46 and the connecting arm 36, they thus provide the operative connection between the control element 29 of the bend control mechanism 30 and the bendable end 14. A more detailed description of their function is given later in connection with FIG. 9.

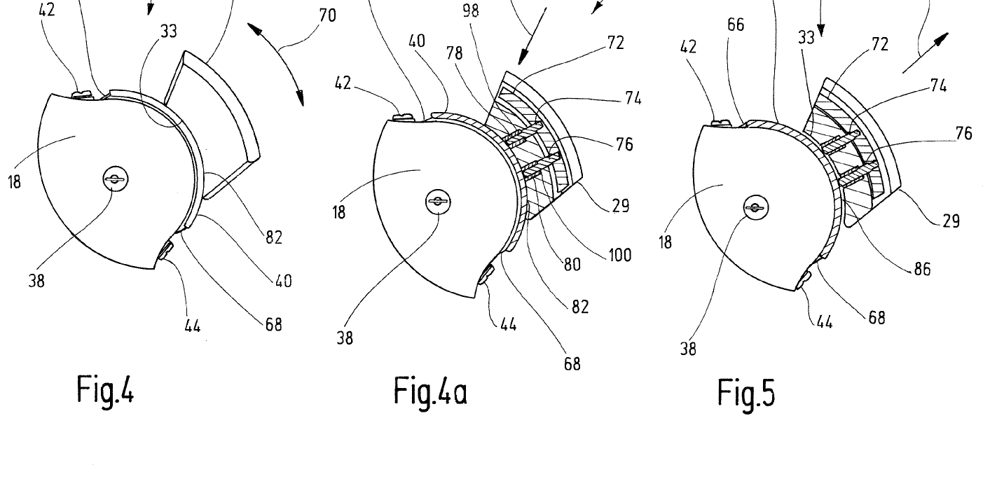

The feature whereby the bend control mechanism 30, and thus the bendable end 14 of the shaft 12, can be locked with the aid of the friction plate 40 will now be described in detail in connection with FIGS. 4 to 8.

FIG. 4 shows that an underside 33 of the control element 29 is in direct contact with the friction plate 40, which is fastened on the handle 18 via angled spring plates 66 and 68 and by means of the screws 42 and 44. The friction plate 40 thus extends at a spacing from the outside of the handle 18 on which it is mounted. A movement of the control element 29 about the pivot axis 38 in the directions of the double arrow 70 is avoided or braked by the frictional contact between the control element 29 and the friction plate 40 on a friction contact face 82.

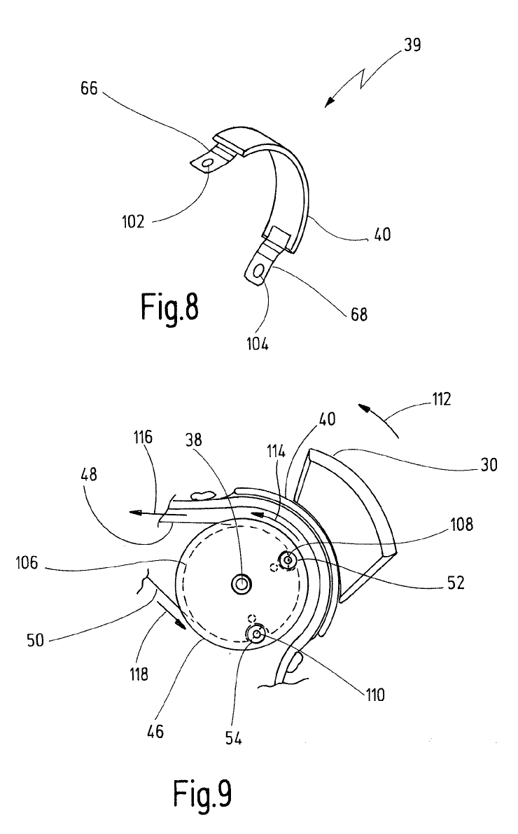

FIG. 4a shows that the bend control mechanism 30 comprises the trigger 72. The latter, as can also be seen in FIG. 1b, is easily accessible to the operating surgeon from the proximal direction. Protruding from the trigger 72 are pins 74, 74′, 76, 76′ (see also FIG. 7) which at the distal end are guided through and held by sleeves 78, 80 in the body of the control element 29. The tips of the pins bear directly on the friction plate 40 and thus provide an operative connection between the trigger 72 and the friction plate 40. By pressing the trigger 72 in the direction of the arrow 84, the pins are moved axially through bores 98, 100 in the body of the control element 29, and they thus press the friction plate 40 in the direction of the handle 18. The friction plate 40 thus moves away from the underside 33 of the control element 29. The friction contact face 82 is thus freed and a gap 86 is formed, as is shown in FIG. 5. The flexibility needed for this change of position of the friction plate 40 is permitted principally by the spring plates 66 and 68, but also by elongate openings 102 and 104, as are shown in FIG. 8.

The position resulting from the actuation of the trigger 72, as shown in FIG. 5, now permits a low-friction movement of the control element 29, as is shown by the double arrow 70 in FIG. 4.

Only the tips of the four pins 74, 74′, 76, 76′ rest on the friction plate 40 and slide with low friction across the surface thereof. For this purpose, they can be made of a low-friction plastic material, for example. It is also possible for a metal main body to be covered by the low-friction material, or for a low-friction tip to be fitted onto a metal stump.

When the operating surgeon now takes his finger, preferably the thumb, off the trigger 72, the tension afforded by the spring plates 66 and 68 means that the friction plate 40 is pressed back against the underside 33 of the control element 29 of the bend control mechanism 30, such that the gap 86 disappears and the friction contact face 82 is once again present. Correspondingly, the pins 74, 74′, 76, 76′ and thus the trigger 72 also undergo a proximal movement in the direction of the arrow 88. In this way, the bend control mechanism 30 is locked in its position again. This can therefore be done steplessly within the pivot range of the control element 29.

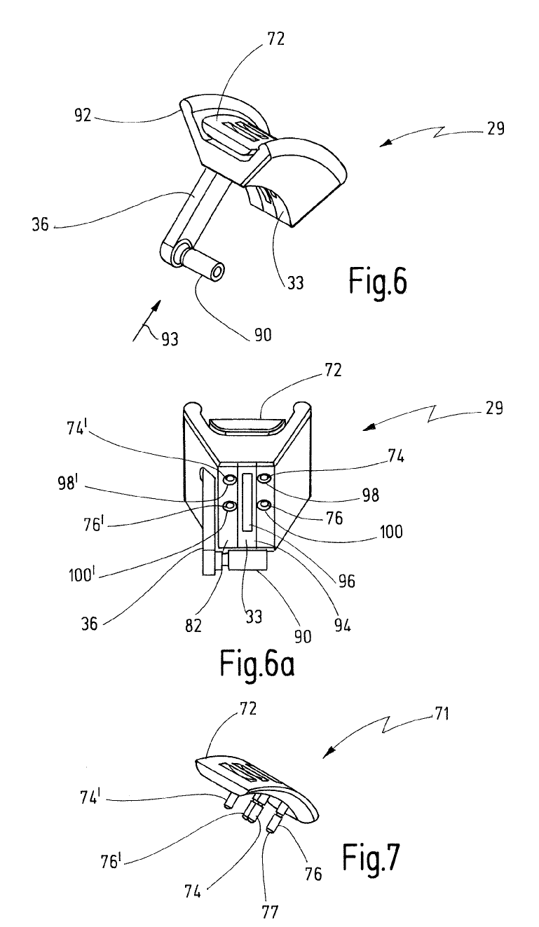

The control element 29 of the bend control mechanism 30 is shown in more detail in FIGS. 6, 6a and 7, in which the trigger 72 and the proximal access to the latter can be clearly seen. The trigger 72 is fastened on a finger-receiving part 92 which is mounted on the pivot axis 38 via the connecting arm 36 and with a pin 90.

FIG. 6a shows the underside 33 which comes into contact with the friction plate 40. In this illustrative embodiment, the trigger 72 is equipped with four pins 74, 74′, 76 and 76′, which extend axially and are movable within the bores 98, 98′, 100 and 100′. Arranged between the two pairs of pins 74, 76 and 74′, 76′, there is a plastic inlet piece 96 which is fastened on the finger-receiving part 92 by a retaining plate 94. This plastic inlet piece 96 serves to increase the friction between the control element 29 and the friction plate 40 and, thereby, reinforce the locking in the desired position.

The trigger 72 with the four pins 74, 74′, 76 and 76′ can be seen clearly in FIG. 7. By virtue of their distally rounded tips 77, the friction as they slide on the friction plate 40 is reduced to a minimum, which facilitates the use of the bend control mechanism 30.

The illustrative embodiment of the friction element 39 with the friction plate 40 shown in FIG. 8 is connected at the opposite ends to the angled spring plates 66 and 68, which both have an elongate opening 102, 104, respectively, and this permits a mobility of the friction plate 40 on the handle 18, according to the above description, in other words towards and away from the handle 18. The angles on the spring plates 66 and 68 provide for the corresponding pressing force and, consequently, for the firm locking between the control element 29 and the handle 18 on which the friction plate 40 is mounted.

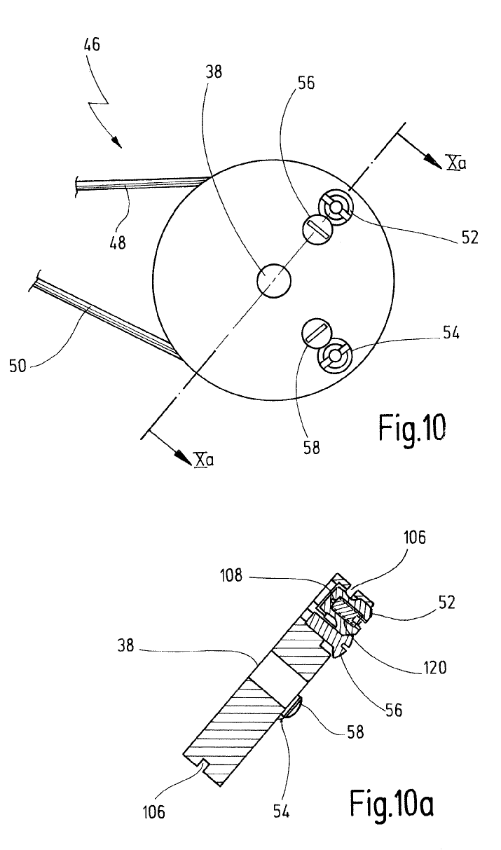

The function of the bend control mechanism 30 will be explained in more detail with reference to FIG. 9 to FIG. 10a, and the fastening of the control wires 48 and 50 on the drum 46 will be described.

FIG. 9 shows the course of the control wires 48 and 50 in the drum 46. The latter comprises a circumferential groove 106 in which the control wires 48 and 50 are guided, in order thereafter to end in bores 108 and 109 of the fastening screws 52 and 54. The control wires 48 and 50 are then mounted firmly on these.

If the control element 29 is now moved in the direction of the arrow 112, the drum 46, because of the above-described operative connection via the connecting arm 36, executes a rotation movement about the pivot axis 38, as is indicated by the direction of the arrow 114. For the control wires 48 and 50 secured on the drum 46, this means that they too execute a movement, specifically with the control wire 48 being pushed into the shaft 112 in the direction of the arrow 116 and with the control wire 50 being drawn out of the shaft in the direction of the arrow 118. As a result of the abovementioned operative connection of the control wires 48 and 50 to the bendable end 14, the angle setting of the latter is consequently changed. This results in a bending movement of the form represented by the bendable end 14′ in FIG. 1.

The opposite movement again leads to a straightening of the shaft 12 or an upward bending movement as seen in FIG. 1. The setting or angle of the bendable end 14 can be locked in any desired position by releasing the trigger 72.

If the arrangement of the drum and of the control element were turned through 90°, this would result, not in the “up-down” bending plane shown in FIG. 1, but in a “left-right” bending plane turned 90° about the shaft axis. The control wires can also be arranged the other way round, in which case, for example, a “forward” displacement of the control element 29 leads to an “upward” bending movement instead of a “downward” bending movement.

FIG. 10a shows the circumferential groove 106. It also shows the bore 108 of the fastening screw 52. Through this, in the example mentioned here, the control wire 48 is inserted into the fastening screw 52 and mounted firmly in this fastening screw by means of a fixing screw 120. The same applies to the fastening screw 54, not shown here in the cross section, and to the control wire 50. The length of the control wires 48 and 50 can then be adjusted by individual rotation of the screws 52 and 54. In one illustrative embodiment, these have mutually different threads for this purpose, such that fastening screw 52 has a right-hand thread and fastening screw 54 has a left-hand thread. After the control wires 48 and 50 have been adjusted, the fastening screws 52 and 54 are fixed by means of the securing screws 56 and 58. These prevent independent rotation of the fastening screws 52 and 54 and thus prevent unwanted adjustment of the control wires 48 and 50.

In FIGS. 11 to 16, the design and assembly of the flexible insert 22 are described.

The insert 22 shown in FIG. 11 has at its distal end a tool 126, in this case two spreadable jaw parts 127, 127′, which tool is operatively connected to a connection piece 130 via a rod-shaped flexible actuating element 128. Mounted proximally behind the tool 126, there are a hood 124 and a screw closure 122 which both serve to fasten the insert 22 on a flexible shaft, e.g. on the flexible shaft 12 from FIG. 1 in an axially immovable manner. As has already been mentioned, the proximal end of the insert 22 has the connection piece 130, which serves, for example, for fastening on the grip part 20 of the medical instrument 10. For this purpose, in this illustrative embodiment, the end has a spherical shape and is arranged proximally behind a portion 131 of smaller diameter on the insert 22.

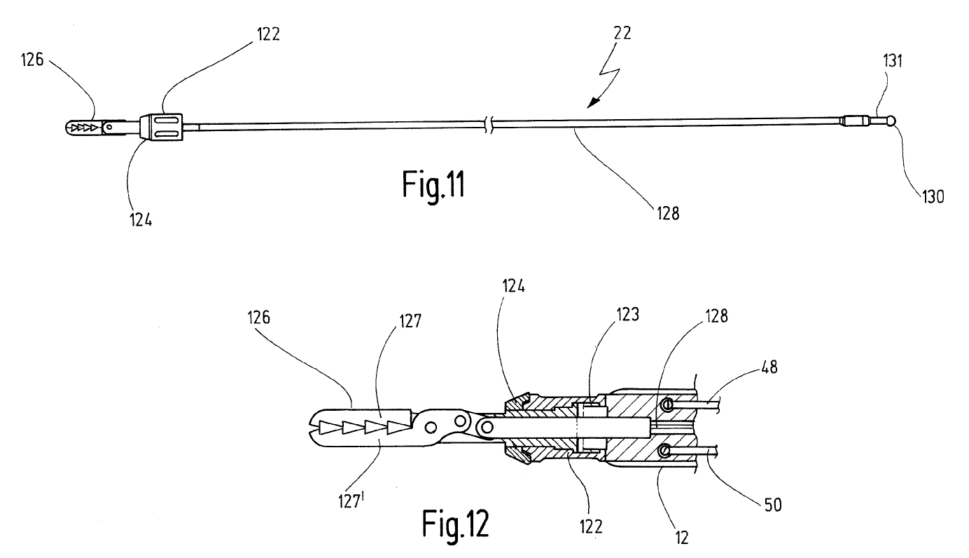

FIG. 12 shows the fastening of the distal end of the insert 22 on the distal end of the shaft 12. The hood 124 located proximally behind the tool 126 is connected firmly to the insert 22. This prevents the screw closure 122 from slipping in a distal direction. This screw closure 122 is for its part then screwed onto an outer thread 123 at the distal end of the shaft 12. For this purpose, the force transmission element 128 is first inserted from the distal direction into the shaft 12. The distal end of the insert 22 is fixed in position by this fastening. A bending of the bendable end 14 then no longer causes the insert 22 to be pushed out from the distal end of the shaft 12.

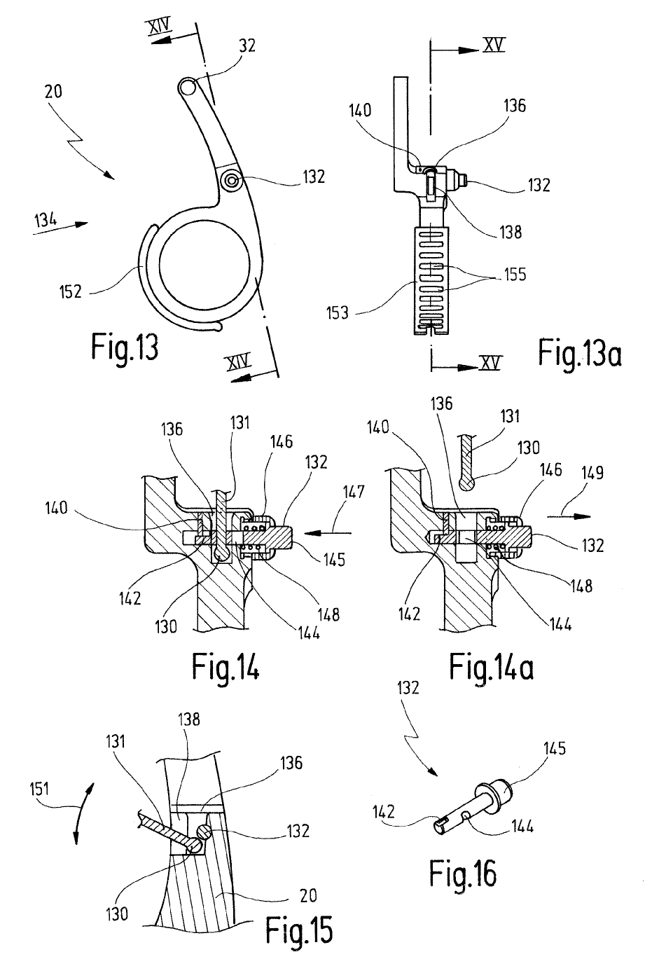

FIGS. 13 to 16 show the grip part 20, the pivot axis 32 thereof and a catch 132 for releasable connection to the proximal end of the insert 22. FIG. 13a shows an opening 136 which opens in the direction of the pivot axis 32 and through which the spherical end of the connection piece 130 is inserted. The portion 131 of small diameter following distally from this on the insert 22 can be guided out laterally from the interior of the grip part 20 via a groove 138 (see FIG. 15). To introduce the end of the insert 22, a catch 132 has to be pressed such that the connection piece 130 can pass the latter. This can be seen from FIGS. 14 and 14a.

The catch 132 shown in FIG. 16 is held by a retainer 146 on the grip part 20. It is further pressed against the edge of this retainer 146 by a spring 148. The position shown in FIG. 14 thus represents the starting position of the catch 132. It will be seen how the connection piece 130, because of its spherical end here, is blocked by the catch 132 and therefore cannot pass upwards, with reference to the drawing, through the opening 136. If the catch 132 is now actuated counter to the direction in which it is pressed by the spring 148, that is to say in the direction of the arrow 147, a recess 144 which is provided on the catch 132, which is located to the right of the connection piece 130 in the view in FIG. 14, moves into a central position of the opening 136, as is shown by way of example in FIG. 14a. This pressing-in can be done via a knob 145 which protrudes laterally outwards past the retainer 146. This recess 144 gives the spherical connection piece 130 enough room to move past this catch 132. In this way, the connection piece 130 can be removed from the retainer in the grip part 20 by way of the opening 136. When the catch 132 is released again, it moves back out again in the direction of the arrow 149 in FIG. 14a. The reason for this is once again the spring 148. At the same time, the recess 144 also moves then.

If the connection piece 130 is then to be fitted back into the retainer of the grip part 20, the catch 132 has to be pressed back in the direction of the arrow 147 in FIG. 14, such that the recess 144 comes to lie once more in the central position, as is shown in FIG. 14a. In this way, the spherical end can be guided past the catch 132 again, and the connection piece 130 can be fastened on the grip part 20 via the opening 136.

FIG. 15 shows how a connection piece 130 is located under the catch 132. An upward movement is not possible. The portion 131 of small diameter on the proximal end of the insert 22 fits through the groove 138, thus permitting mobility in the direction of the double arrow 151. This freedom of movement is needed in the movement of the grip part 20.

To avoid a rotation of the catch 132 pivotable about the longitudinal axis, and thus also to avoid a rotation of the recess 144, an axial groove 142 is formed at the distal end of the catch 132. This groove 142 also serves as an abutment for the displacement movement. This is shown in FIGS. 14 and 14a, and also in the perspective view in FIG. 16. A pin 140 now ends in this groove 142 upon fastening in the grip part 20 and, although it prevents undesired rotation about the longitudinal axis of the catch 132, it nevertheless permits an axial mobility of the catch 132 in the direction of the arrows 147 and 149.

In FIGS. 17 to 20, the lock connection formed by the lock 24 on the grip part 20 is shown in detail.

The lock 24 is mounted in a recess 163 on the handle 18 so as to be pivotable about a pivot axis 150. In this illustrative embodiment, this lock 24, by contact with the grip part 20, can suppress the movement of the grip part 20 in the distal direction. For this purpose, the lock 24 is pressed in the direction of the grip part 20 by the pretensioning afforded by a spring plate 166.

For this purpose, the lock 24, on its side directed towards the grip part 20, has locking teeth 174 which come into engagement with a locking pin 160 on the grip part 20. The inclination of the flanks of the locking teeth 174 in the direction of the handle 18 permits a movement of the grip part 20 in the direction of the handle 18, but blocks this in the opposite direction.

If the lock connection is to be released briefly, the lock 24 is pivoted in the direction of the arrow 170, preferably by actuation via the arc-shaped attachment 25, which leads to an end position as shown in FIG. 18. Because of the pretensioning, the lock 24, when released, is brought back again to the grip part 20 in the direction of the arrow 172.

In order to deactivate the lock connection for a period of time, a detent 152 is provided on the grip part 20.

The detent 152 is designed as a curved element, in the illustrative embodiment shown here as a curved strip 153 (see also FIG. 13a) whose curvature is adapted to the curvature of the outer face of the ring section 23 of the grip part 20.

Recesses or punches 155 in the strip 153 increase its grip.

As can be seen in FIG. 17, this detent 152 can be brought between grip part 20 and lock 24. In this case, the lock connection is deactivated and the grip part 20 is movable freely in both directions. For this purpose, the detent 152 has a rounded nose 157, which can run in both directions over the teeth 174. This corresponds to a second position of the detent 152. In order now to reactivate the lock connection, the detent 152 can be pushed in the direction of a locking pin 162. This corresponds to a first position of the detent 152. A cover 158 is provided on both sides of the strip 153. This cover 158 conceals a guide pin 156 which extends transversely in the detent and which runs in guide grooves 154 on both sides of the ring section 23. The covers 158 themselves can be fastened on the detent 152 by pins (not shown here). Accordingly, the detent 152 extends through a circular movement, as is defined by the shape of the ring section 23 of the grip part 20, and thus ends in a position as shown in FIG. 18. In this way, a locking pin 160 previously blocked by the detent 152 now lies free and can come into engagement with the teeth 174 of the lock 24.

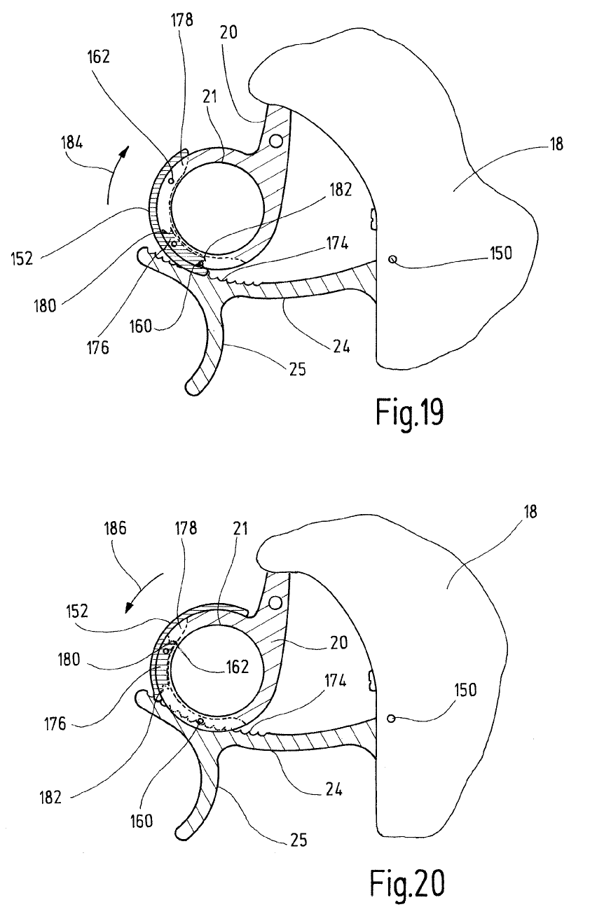

As is shown in FIGS. 19 and 20, an outer groove 178 is cut into the circumference of the grip part 20. A step 176 of the detent 152 projecting in the radial direction of the ring section 23 can be moved in this groove 178, which step 176 is arranged centrally on the detent 152. Spring clips 180 and 182 are arranged respectively at each end of this step 176. They are able to engage in the locking pins 160 and 162, respectively, in accordance with the position of the detent 152 and thus prevent a simple reciprocating sliding of the detent 152. The latter is thus held in the respective positions.

FIG. 19 shows, in this connection, the second position of the detent 152, in which the lock connection is deactivated. The spring clip 182 of the step 176 on the detent 152 is engaged in the locking pin 160 and thus blocks the contact between the locking teeth 174 and the locking pin 160. A movement of the detent 152 in the direction of the arrow 184 would finally end in the first position, as is shown in FIG. 20. The spring clip 180 located on the step 176 is engaged in the locking pin 162, and the detent 152 is thus fixed in this position. The locking pin 160 thus lies free and is able to hook into the teeth 174 of the lock 24.

By contrast, a proximal movement of the grip part 20, which would lead for example to a closing of the jaw parts 127, 127′, is again possible via the lock 24.

The lock connection can now be deactivated again by moving the detent 152 analogously to what has been stated above in the direction of the arrow 186, preferably after the lock 24 has been lowered, in accordance with the description of FIGS. 17 and 18.

As can be seen in FIG. 1a, the operator can hold the instrument 10 via the handle 18. The trigger 72 can be pressed by the thumb and the control element 29 then displaced. This causes a corresponding bending of the bendable end 14 of the shaft. Release of the trigger 72 stops the bendable end 14 in the corresponding position.

A movement of the grip part 20, e.g. by the inserted index finger, permits the opening and closing of the jaw parts 127, 127′ via the insert 22 in any desired angled position of the bendable end 14 of the shaft 12.

When the lock function is deactivated, the movement of the grip part 20 is possible in both directions of pivoting.

When the lock function is activated, this can be quickly obtained by pivoting the lock 24 with the middle finger via the arc-shaped attachment 25.

The operator is thus able to manoeuvre the medical instrument 10 easily and safely and in a highly ergonomic manner.

What is claimed is:

1. A medical instrument comprising
a flexible shaft having a bendable distal end,
a handle arranged at a proximal end of said flexible shaft,
a flexible insert extending along said flexible shaft, said flexible insert having a tool at its distal end and a connection piece at its proximal end, said connection piece being releasably connected to a grip part mounted on said handle, said grip part being mounted pivotally about a pivot axis, wherein
said flexible insert being mounted in an axially immovable manner at said bendable distal end of said shaft, and wherein said connection piece of said flexible insert being connected to said grip part in an articulated manner.

2. The medical instrument of claim 1, wherein said distal end of said insert is fastened on said flexible shaft by a screw fastening.

3. The medical instrument of claim 1, wherein said distal end of said insert is fastened on said flexible shaft by a lock mechanism.

4. The medical instrument of claim 1, wherein said connection piece has a spherical shape.

5. The medical instrument of claim 1, wherein said connection piece can be held in an articulated manner in said grip part by means of a catch.

6. The medical instrument of claim 5, wherein said catch can be moved from a locking engagement with said connection piece to a non-locking position.

7. The medical instrument of claim 6, wherein said catch is designed as an axial pin extending traverse to a longitudinal direction of said medical instrument and which axial pin has a recess through which said connection piece can be moved in said non-locking position.

8. The medical instrument of claim 1, wherein said movable grip part has an opening via which said connection piece can be pushed into said grip part.

9. The medical instrument of claim 7, wherein said catch is held on said grip part by a retainer and is pretensioned against said retainer by a spring.

10. The medical instrument of claim 9, wherein said catch has a knob which protrudes from said retainer and via which knob said catch can be moved from an outside.

11. The medical instrument of claim 8, wherein a portion of said insert arranged distally in front of said connection piece and having a smaller diameter can be inserted into a groove connected to said opening and can be guided laterally out of said grip part via said groove, said connection piece being larger than a width of said groove.

12. The medical instrument of claim 6, wherein said catch has an axial groove into which a pin arranged in said grip part can be inserted.
